SEQUENCE LISTING (141 sequences)

SEQ ID NO: 1 - protein, 131 aa, Mus musculus
  VARIANT 101, 102, 103, 109, 110, 111: Xaa = Any Amino Acid
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTXXXCSGGSXXXYYYYGMDVWGQGTTVTVSSA

SEQ ID NO: 2 - protein, 131 aa, Mus musculus
EVQLVESGGGLVKPGGSLRLSCAASGFTFNNAWMSWVRQAPGKGLEWVGRIKSKIDGGTTDYAAPVKGRFTMSRDDSKNTLYLQMNSLKTEDTAVYYCTTDEDCSGGSCFFYHYYLMDVWGQGTTVTVSSA

SEQ ID NO: 3 - protein, 128 aa, Mus musculus
  VARIANT 99, 100, 112: Xaa = Any Amino Acid
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARXXYDYVWGSYRYTXAFDIWGQGTMVTVSSA

SEQ ID NO: 4 - protein, 128 aa, Mus musculus
QVQLVESGGGVVQPGRSLRLSCAASGFSFINYGMHWVRQAPGKGLEWVAVIWNDGRSKYYADSVKGRFTISRDNSKNTLGLQMNSLRAEDTAVYYCAREDYDYIWESFPYTGAFDIWGQGTMVTVSSA

SEQ ID NO: 5 - protein, 128 aa, Mus musculus
QVQLVESGGGVVQPGRSLRLSCAASGFTFSSYGMHWVRQAPGKGLEWVAVIWNDGSYKYYADSVKGRFTISRDNSKNTLYLQMNSLRAEDTAVYYCVREDYDYDWMIDRYTGAFDLWGQGTMVTVSSA

SEQ ID NO: 6 - protein, 127 aa, Mus musculus
  VARIANT 97, 98, 99, 106, 107: Xaa = Any Amino Acid
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCXXXVDIVATXXYYYYGMDVWGQGTTVTVSSA

SEQ ID NO: 7 - protein, 127 aa, Mus musculus
  VARIANT 97, 98: Xaa = Any Amino Acid
EVQLLESGGGLVQPGGSLRLSCAASGFTFSNYAMSWVRQAPGKGLEWVSVISGSGGNTYYADSVRGRFTISRDNSKNTLYLQMNSLRAEDTAVYYCXXHVDIVATMAYFDYVMDVWGQGTTVTVSSA

SEQ ID NO: 8 - protein, 126 aa, Mus musculus
  VARIANT 101, 112: Xaa = Any Amino Acid
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTXMITFGGVIVIXDYWGQGTLVTVSSA

SEQ ID NO: 9 - protein, 126 aa, Mus musculus
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTEMITFGGVIVIPDYWGQGTLVTVSSA

SEQ ID NO: 10 - protein, 126 aa, Mus musculus
  VARIANT 101: Xaa = Any Amino Acid
EVQLVESGGGLVKPGGSLRLSCAASGFTFSNALMSWVRQAPGKGLEWVGRIKSKTDGGTIDYAAPVKGRFTISRDDSKNTLYLQMNSLKTEDTAVYYCTTXMITFGGVIVIPDYWGQGTLVTVSSA

SEQ ID NO: 11 - protein, 126 aa, Mus musculus
  VARIANT 112: Xaa = Any Amino Acid
EVQLVESGGGLVKPGGSLRLSCAASGFTFSNAWMSWVRQAPGKGLEWVGRIKSKTDGGTIDYVAPVKGRFTISRDDSKNTLYLQMNSLKTEDTAVYYCTTEVITFGGIIVDXDYWGQGTLVTVSSA

SEQ ID NO: 12 - protein, 124 aa, Mus musculus
  VARIANT 99, 100, 106, 107, 108: Xaa = Any Amino Acid
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARXXGIAVAXXXYFDYWGQGTLVTVSSA

SEQ ID NO: 13 - protein, 124 aa, Mus musculus
QVQLVESGGGVVQPGRSLRLSCAASGFTFNTYVMHWVRQAPGKGLEWVAVIWYDGSNKKYADSVKGRFTISRANSKNTLYLQMNSLRAEDTAVYYCARDGGRTVADPYYFDYWGQGTLVTVSSA

SEQ ID NO: 14 - protein, 124 aa, Mus musculus
QVQLVESGGGVVQPGRSLRLSCAASGFTFSSYGMHWVRQAPGKGLEWVAVIWYDGSNKNYADSVKGRFTISRGNSKNTLYLQMNSLRAEDTAVYYCARDGGIAVADPYYFDYWGQGTLVTVSSA

SEQ ID NO: 15 - protein, 124 aa, Mus musculus; identical to SEQ ID NO: 13

SEQ ID NO: 16 - protein, 124 aa, Mus musculus; identical to SEQ ID NO: 13

SEQ ID NO: 17 - protein, 125 aa, Mus musculus
  VARIANT 98, 99, 100, 101, 102, 104, 105: Xaa = Any Amino Acid
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAXXXXXGXXYYYYGMDVWGQGTTVTVSSA

SEQ ID NO: 18 - protein, 125 aa, Mus musculus
  VARIANT 98: Xaa = Any Amino Acid
EVQLVESGGGLVKPGGSLRLSCAASGFTFSSYTMNWVRQAPGKGLEWVSSISSSSSYIYYADSVKGRFTISRDNAKNSLYLQMNSLRAEDTAVYYCAXDGGIGFYYYYYGMDVWGQGTTVTVSSA

SEQ ID NO: 19 - protein, 125 aa, Mus musculus
  VARIANT 98, 101, 102, 104: Xaa = Any Amino Acid
EVQLVESGGGLVKPGGSLRLSCAASGFTFSSYCMNWVRQTPGKGLEWVSSISSSSYYIYYADSVKGRFTISRDNAKNSLYLQMNSLRAEDTAVYYCAXDIXXGXGGDYYGMDVWGQGTTVTVSSA

SEQ ID NO: 20 - protein, 126 aa, Mus musculus
  VARIANT 99, 100, 101, 102, 103, 104, 105: Xaa = Any Amino Acid
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKXXXXXXXYYYYYGMDVWGQGTTVTVSSA

SEQ ID NO: 21 - protein, 126 aa, Mus musculus
EVQLLESGGGLVQPGGSLRLSCAASGFTFSSYAMSWVRQAPGKGLEWVSSISGSGAYTYYADSVKGRFTISRDNSKNTLYLQMNSLRAEDTAVYYCAKDLGEDEDYYDYYGVDVWGQGTTVTVSSA

SEQ ID NO: 22 - protein, 113 aa, Mus musculus
  VARIANT 100, 101, 102: Xaa = Any Amino Acid
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFXXXFGQGTKLEIKR

SEQ ID NO: 23 - protein, 113 aa, Mus musculus
DIVMTQTPLSSPVTLGQPASISCRSSQSLVHNDGNTYLSWLQQRPGQPPRLLIYKISYRFSGVPDRFTGSGAGTDFTLKISRVEPEDVGIYYCIQTTQFPCSFGQGTKLEIKR

SEQ ID NO: 24 - protein, 113 aa, Mus musculus
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPITFGQGTRLEIKR

SEQ ID NO: 25 - protein, 113 aa, Mus musculus
DIVMTQTPLSLSVTPGQPASISCRSSQSLLHSDGETCLHWYLQKPGQPPQLLIYEVSNRFSGVPYRFSGSGSGTDFTLKISRVEAEDVGVYYCMQSIQLPITFGQGTRLEIKR

SEQ ID NO: 26 - protein, 113 aa, Mus musculus
DIVLTQTPLSLSVTPGQPASISCRSTQSLLHSDGETCLHWYLQKPGQPPQLLIYEVSYQFSGVPDRFSGSGSGTDFTLKISRVEAEDVGVYYCMQSIQRPITFGQGTRLEIKR

SEQ ID NO: 27 - protein, 109 aa, Mus musculus
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKR

SEQ ID NO: 28 - protein, 109 aa, Mus musculus
EIVLTQSPGTLSLSPGERASLSCRASQSVSSSYLAWYQHKPGQAPRLLIYGASSRATGIPDRFSGSGSGTDFTLTISRLEPEDFAVYYCQQYGSSPWTFGQGTKVEVKR

SEQ ID NO: 29 - protein, 112 aa, Mus musculus
  VARIANT 101: Xaa = Any Amino Acid
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPXFGGGTKVEIKR

SEQ ID NO: 30 - protein, 112 aa, Mus musculus
  VARIANT 101: Xaa = Any Amino Acid
DIVMTQSPLSLPVTPGEPASISCRSSQSLLHRNGYNYLEWYLQKPGQSPQLLIYLGSNRASGVPDRFSGSGSGTDFTLKISRVEAEDVGFYYCMQALQTPXFGGGTKVEIKR

SEQ ID NO: 31 - protein, 113 aa, Mus musculus
  VARIANT 100, 101: Xaa = Any Amino Acid
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLXXTFGGGTKVEIKR

SEQ ID NO: 32 - protein, 113 aa, Mus musculus
DIVMTQTPLSLTVTPGQPASISCKSSQSLLHSDGKTYLYWYLQKPGQPPQFLIYEVSNRFSGVPDRFSGSGSGTDFTLKISRVEAEDAGVYYCMQSIQFPLTFGGGTKVEIKR

SEQ ID NO: 33 - protein, 113 aa, Mus musculus
DIVMTQTPLSLSVTPGQPASISCKSSQSLLHSDGKTYLYWYLQRPGQPPQFLIYEVSNRFSGVPDRFSGSGSGTDFTLKISRVEAEDVGIYYCMQSIQLPLTFGGGTKVELKR

SEQ ID NO: 34 - protein, 113 aa, Mus musculus
DIVMTQTPLSLSVTPGQPASISCKSSQSLLHSDGKTYLYWYLQRPGQPPQFLIYEVSNRFSGVPDRFSGSGSGTDFTLKISRVEAEDVGVYYCMQSIQLPLTFGGGTKVEVKR

SEQ ID NO: 35 - protein, 113 aa, Mus musculus; identical to SEQ ID NO: 34

SEQ ID NO: 36 - protein, 108 aa, Mus musculus
DIQMTQSPSSLSASVGDRVTITCRASQGISNYLAWYQQKPGKVPKLLIYAASTLQSGVPSRFSGSGSGTDFTLTISSLQPEDVATYYCQKYNSAPWTFGQGTKVEIKR

SEQ ID NO: 37 - protein, 108 aa, Mus musculus
DIQMTQSPSSLSASVGDRVTITCRTSQGINNYLAWYQQKPGKVPKLLIYAASTLRSGVPSRFSGSGSGTDFTLTISSLQPEDVATYYCQKYNSAPWTFGQGTKVEIKR

SEQ ID NO: 38 - protein, 108 aa, Mus musculus; identical to SEQ ID NO: 36

SEQ ID NO: 39 - protein, 108 aa, Mus musculus
DIQMTQSPSSLSASVGDRVTITCRASQGISNYLAWYQQKPGKVPELLIYAASTLQSGVPSRFSGSGSGTDFTLTISSLQPEDVATYYCQKYDSAPWTFGQGTKVEIKR

SEQ ID NO: 40 - protein, 108 aa, Mus musculus
DIQMTQSPSSLSASVGDRVTITCRASQGISNYLAWYQQKPGKVPKLLIYAASTLQSGVPSRFSGSGSGTDFTLTISSLQPEDVATYYCQKYNSAPFTFGPGTKVDIKR

SEQ ID NO: 41 - protein, 108 aa, Mus musculus
DIQMTQSPSSLSASVGDRVTITCRASQGISNDLAWYQQKPGKIPNLLIYAASTLQSGVPSRFSGSGSGTDFTLTIGSLQPEDVATYYCQKYNSAPFTFGPGTKVDIKR

SEQ ID NO: 42 - protein, 112 aa, Mus musculus
  VARIANT 99, 100: Xaa = Any Amino Acid
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQXXTFGQGTRLEIKR

SEQ ID NO: 43 - protein, 112 aa, Mus musculus
DIVMTQSPLSLPVTPGEPASISCRSSQSLLHSNGYNYLDWYLQKPGQSPQLLIYLGSNRASGVPDRFSGSESDTDFTLKISRVEAEDVGVYYCMQALQSPTFGQGTRLEIKR

SEQ ID NO: 44 - DNA, 13 bp, Mus musculus
aagacgggggcat

SEQ ID NO: 45 - DNA, 20 bp, Mus musculus
gtggatatagtggctacgat

SEQ ID NO: 46 - DNA, 17 bp, Mus musculus
gggtatagcagtggctg

SEQ ID NO: 47 - DNA, 19 bp, Mus musculus
attgtagtggtggtagctg

SEQ ID NO: 48 - DNA, 25 bp, Mus musculus
tgattacgtttgggggaattatcgt

SEQ ID NO: 49 - DNA, 35 bp, Mus musculus
attatgattacgattggatgattgatcgttatacc

SEQ ID NO: 50 - DNA, 17 bp, Mus musculus
gggtagaacagtggctg

SEQ ID NO: 51 - DNA, 32 bp, Mus musculus
atgattacgtttgggggagttatcgttatacc

SEQ ID NO: 52 - DNA, 35 bp, Mus musculus
attatgattacatttgggagagttttccttatacc

SEQ ID NO: 53 - DNA, 17 bp, Mus musculus
gggtagaacagtggctg

SEQ ID NO: 54 - DNA, 17 bp, Mus musculus
gggtagaacagtggctg

SEQ ID NO: 55 - DNA, 33 bp, Mus musculus
tatgattacgtttgggggagttatcgttatacc

SEQ ID NO: 56 - DNA, 13 bp, Mus musculus
cagtggcgggtac

SEQ ID NO: 57 - DNA, 17 bp, Mus musculus
tctaggtgaggatgagg

SEQ ID NO: 58 - protein, 10 aa, Mus musculus
GFTFSNYAMS

SEQ ID NO: 59 - protein, 10 aa, Mus musculus
GFTFSSYAMS

SEQ ID NO: 60 - protein, 10 aa, Mus musculus
GFTFSSYGMH

SEQ ID NO: 61 - protein, 10 aa, Mus musculus
GFTFNNAWMS

SEQ ID NO: 62 - protein, 10 aa, Mus musculus
GFTFSNAWMS

SEQ ID NO: 63 - protein, 10 aa, Mus musculus
GFTFSSYGMH

SEQ ID NO: 64 - protein, 10 aa, Mus musculus
GFTFNTYVMH

SEQ ID NO: 65 - protein, 10 aa, Mus musculus
GFTFSNAWMS

SEQ ID NO: 66 - protein, 10 aa, Mus musculus
GFSFINYGMH

SEQ ID NO: 67 - protein, 10 aa, Mus musculus
GFTFNTYVMH

SEQ ID NO: 68 - protein, 10 aa, Mus musculus
GFTFNTYVMH

SEQ ID NO: 69 - protein, 10 aa, Mus musculus
GFTFSNALMS

SEQ ID NO: 70 - protein, 10 aa, Mus musculus
GFTFSSYTMN

SEQ ID NO: 71 - protein, 10 aa, Mus musculus
GFTFSSYCMN

SEQ ID NO: 72 - protein, 17 aa, Mus musculus
VISGSGGNTYYADSVRG

SEQ ID NO: 73 - protein, 17 aa, Mus musculus
SISGSGAYTYYADSVKG

SEQ ID NO: 74 - protein, 17 aa, Mus musculus
VIWYDGSNKNYADSVKG

SEQ ID NO: 75 - protein, 19 aa, Mus musculus
RIKSKIDGGTTDYAAPVKG

SEQ ID NO: 76 - protein, 19 aa, Mus musculus
RIKSKTDGGTTDYVAPVKG

SEQ ID NO: 77 - protein, 17 aa, Mus musculus
VIWNDGSYKYYADSVKG

SEQ ID NO: 78 - protein, 17 aa, Mus musculus
VIWYDGSNKKYADSVKG

SEQ ID NO: 79 - protein, 19 aa, Mus musculus
RIKSKTDGGTTDYAAPVKG

SEQ ID NO: 80 - protein, 17 aa, Mus musculus
VIWNDGRSKYYADSVKG

SEQ ID NO: 81 - protein, 17 aa, Mus musculus
VIWYDGSNKKYADSVKG

SEQ ID NO: 82 - protein, 17 aa, Mus musculus
VIWYDGSNKKYADSVKG

SEQ ID NO: 83 - protein, 19 aa, Mus musculus
RIKSKTDGGTIDYAAPVKG

SEQ ID NO: 84 - protein, 17 aa, Mus musculus
SISSSSSYIYYADSVKG

SEQ ID NO: 85 - protein, 17 aa, Mus musculus
SISSSSYYIYYADSVKG

SEQ ID NO: 86 - protein, 17 aa, Mus musculus
HVDIVATMAYFDYVMDV

SEQ ID NO: 87 - protein, 16 aa, Mus musculus
DLGEDEDYYDYYGVDV

SEQ ID NO: 88 - protein, 14 aa, Mus musculus
DGGIAVADPYYFDY

SEQ ID NO: 89 - protein, 19 aa, Mus musculus
DEDCSGGSCFFYHYYGLDV

SEQ ID NO: 90 - protein, 14 aa, Mus musculus
EVITFGGIIVDFDY

SEQ ID NO: 91 - protein, 18 aa, Mus musculus
EDYDYDWMIDRYTGAFDL

SEQ ID NO: 92 - protein, 14 aa, Mus musculus
DGGRTVADPYYFDY

SEQ ID NO: 93 - protein, 14 aa, Mus musculus
EMITFGGVIVIPDY

SEQ ID NO: 94 - protein, 18 aa, Mus musculus
EDYDYIWESFPYTGAFDI

SEQ ID NO: 95 - protein, 14 aa, Mus musculus
DGGRTVADPYYFDY

SEQ ID NO: 96 - protein, 14 aa, Mus musculus
DGGRTVADPYYFDY

SEQ ID NO: 97 - protein, 14 aa, Mus musculus
DMITFGGVIVIPDY

SEQ ID NO: 98 - protein, 15 aa, Mus musculus
DGGIGFYYYYYGMDV

SEQ ID NO: 99 - protein, 18 aa, Mus musculus
DISGGYGGDYYYYYGMDV

SEQ ID NO: 100 - protein, 11 aa, Mus musculus
RTSQGINNYLA

SEQ ID NO: 101 - protein, 16 aa, Mus musculus
KSSQSLLHSDGKTFLF

SEQ ID NO: 102 - protein, 11 aa, Mus musculus
RASQDISNYLV

SEQ ID NO: 103 - protein, 16 aa, Mus musculus
KSSQSLLHSDGETFLF

SEQ ID NO: 104 - protein, 12 aa, Mus musculus
RASQSVRTNYLV

SEQ ID NO: 105 - protein, 16 aa, Mus musculus
KSSQSLLHSDGKTFLF

SEQ ID NO: 106 - protein, 16 aa, Mus musculus
RSSQSLVHNDGNTYLS

SEQ ID NO: 107 - protein, 11 aa, Mus musculus
RASQGISNYLA

SEQ ID NO: 108 - protein, 16 aa, Mus musculus
RSSQSLLHSDGETCLH

SEQ ID NO: 109 - protein, 16 aa, Mus musculus
KSSQSLLHSDGKTFLF

SEQ ID NO: 110 - protein, 16 aa, Mus musculus
RSSQSLLHSNGYNYLD

SEQ ID NO: 111 - protein, 11 aa, Mus musculus
RASQGISNDLA

SEQ ID NO: 112 - protein, 16 aa, Mus musculus
RSSQSLLHRNGYNYLE

SEQ ID NO: 113 - protein, 16 aa, Mus musculus
KSTQSLLHSDGKTYLY

SEQ ID NO: 114 - protein, 7 aa, Mus musculus
AASTLRS

SEQ ID NO: 115 - protein, 7 aa, Mus musculus
EVSNRFS

SEQ ID NO: 116 - protein, 7 aa, Mus musculus
AASTLQS

SEQ ID NO: 117 - protein, 7 aa, Mus musculus
EVSNRFS

SEQ ID NO: 118 - protein, 7 aa, Mus musculus
GASSRAP

SEQ ID NO: 119 - protein, 7 aa, Mus musculus
EVSNRFS

SEQ ID NO: 120 - protein, 7 aa, Mus musculus
KISYRFS

SEQ ID NO: 121 - protein, 7 aa, Mus musculus
AASTLQS

SEQ ID NO: 122 - protein, 7 aa, Mus musculus
EVSNRFS

SEQ ID NO: 123 - protein, 7 aa, Mus musculus
EVSNRFS

SEQ ID NO: 124 - protein, 7 aa, Mus musculus
LGSNRAS

SEQ ID NO: 125 - protein, 7 aa, Mus musculus
AASTLQS

SEQ ID NO: 126 - protein, 7 aa, Mus musculus
LGSNRAS

SEQ ID NO: 127 - protein, 7 aa, Mus musculus
EVSYQFS

SEQ ID NO: 128 - protein, 9 aa, Mus musculus
QKYNSAPWT

SEQ ID NO: 129 - protein, 9 aa, Mus musculus
MQSIQLPLT

SEQ ID NO: 130 - protein, 9 aa, Mus musculus
QKYDSAPWT

SEQ ID NO: 131 - protein, 9 aa, Mus musculus
MQSIQFPLT

SEQ ID NO: 132 - protein, 9 aa, Mus musculus
QQYGTSPWA

SEQ ID NO: 133 - protein, 9 aa, Mus musculus
MQSIQLPLT

SEQ ID NO: 134 - protein, 9 aa, Mus musculus
IQTTQFPCS

SEQ ID NO: 135 - protein, 9 aa, Mus musculus
QKYNSAPWT

SEQ ID NO: 136 - protein, 9 aa, Mus musculus
MQSIQLPIT

SEQ ID NO: 137 - protein, 9 aa, Mus musculus
MQSIQLPLT

SEQ ID NO: 138 - protein, 8 aa, Mus musculus
MQALQSPT

SEQ ID NO: 139 - protein, 9 aa, Mus musculus
QKYNSAPFT

SEQ ID NO: 140 - protein, 8 aa, Mus musculus
MQALQIPT

SEQ ID NO: 141 - protein, 9 aa, Mus musculus
MQSIQRPIT

(19) United States
(12) Patent Application Publication
(10) Pub. No.: US 2005/0013809 A1
(43) Pub. Date: Jan. 20, 2005

(54) Antibodies against drugs of abuse

(76) Inventors: Samuel M. Owens, Little Rock, AR (US); Frank Ivy Carroll, Durham, NC (US); Philip Abraham, Cary, NC (US); Melinda G. Gunnell, Conway, AR (US); Mary Haak-Frendscho, Newark, CA (US); Xiao Feng, Union City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/725,962

(22) Filed: Dec. 2, 2003

Related U.S. Application Data
(60) Provisional application No. 60/430,717, filed on Dec. 2, 2002.

(51) Int. Cl. A61K 39/395; C12N 5/06; C07K 16/18

(52) U.S. Cl. 424/130.1; 530/387.1; 435/326

(57) ABSTRACT

The present invention is related to antibodies directed to various drugs of abuse and uses of such antibodies. In preferred embodiments, the drugs of abuse are amphetamine, methamphetamine, or phencyclidine (PCP). In particular, in accordance with the present invention, there are provided fully man monoclonal antibodies directed to drugs of abuse. Nucelotide sequences encoding, and amino acid sequences comprising, heavy and light chain immunoglobulin molecules, particularly sequences corresponding to contiguous heavy and light chain sequences spanning the framework regions and/or complementarity determining regions (CDR's), specifically from FR1 through FR3 or CDR1 through CDR3, are provided. Hybridomas or other cell lines expressing such immunoglobulin molecules and monoclonal antibodies are also provided.

### RELATED APPLICATIONS

This application claims priority to U.S. Provisional Application No. 60/430,717 which was filed on Dec. 2, 2002, and is hereby incorporated by reference in its entirety.

### Governmental Support

This invention was made with Government support by Grant Nos. DA 07610, DA 14361, and DA 11560, awarded by the National Institute on Drug Abuse. The Government has certain rights in this invention.

### BACKGROUND OF THE INVENTION

1. Field of the Invention

The present invention is related to antibodies capable of binding to drugs of abuse and use of such antibodies. Of particular interest are various antibodies against amphetamine, methamphetamine, and phencyclidine (PCP). In accordance with the present invention, there are provided fully human monoclonal antibodies directed to drugs of abuse. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDR's), specifically from FR1 through FR3 or CDR1 through CDR3, are provided. Hybridomas or other cell lines expressing such immunoglobulin molecules and monoclonal antibodies are also provided.

2. Description of the Related Art

Drug addiction, drug abuse, and drug overdose are problems which afflict many individuals in the United States and in foreign countries. These “drugs of abuse” or “drugs” refer to chemical agents which are either ingested or otherwise consumed by an individual and which may induce adverse health consequences. Drugs of abuse may or may not be regulated by government entities. The most common drugs of abuse include marijuana, cocaine, amphetamines, phencyclidine (PCP), heroin, hallucinogens, alcohol, nicotine, prescription medications, steroids, and inhalants.

Although the motivations for use and pharmacokinetics can vary widely from one drug to the next, most drugs of abuse are taken for the physical pleasure or heightened abilities they provide to the user. Amphetamines are one example of such a drug, and which is often taken by drug users for both reasons.

Amphetamines belong to a group of drugs called psychostimulants which stimulate the central nervous system. In addition to amphetamine itself, there are numerous derivatives in the amphetamine family including methamphetamine and methoxylated amphetamines. In general, amphetamines increase a person's heart rate and respiration rate, increase blood pressure, dilate the pupils of the eyes, and decrease appetite. Repeated use of amphetamines can lead to addiction and numerous health problems, including irregular heartbeat, damage to internal organs, psychosis, physical collapse, and in some cases, death. Amphetamines can be physically and psychologically addictive and users who abruptly stop using them often experience signs of addiction, such as fatigue, long periods of sleep, irritability, and depression.

A frequently used derivative of amphetamine is methamphetamine. This drug is also a stimulant and can cause an increase in heart and respiratory rates, along with elevated blood pressure, dilated pupils and decrease in appetite. Users may also experience sweating, headache, blurred vision, dizziness, sleeplessness and anxiety. Very high doses can cause rapid or irregular heartbeat, tremors, loss of coordination and physical collapse. When used in injection form there is a sudden increase in blood pressure that can result in stroke, very high fever or heart failure. Users of this drug report feeling restless, anxious and have mood swings. With increased doses comes increased effects. Users, over a long period of time, can develop an amphetamine psychosis which could include hallucinations, delusions and paranoia.

Another drug of abuse is phencyclidine, or PCP. This drug interrupts the functions of the neocortex, the part of the brain which controls intellect and instinct. PCP also blocks pain receptors which can lead to violent PCP episodes which result in self-inflicted injuries. The effects of PCP on an individual vary, but most frequently they include a sense of distance and estrangement. Time and body movements slow down and muscular coordination worsens along with the senses being dulled. Speech is blocked and incoherent. After chronic use, a person can become paranoid, violent and suffer from hallucinations. Large doses of this hallucinogenic drug can produce convulsions, coma, as well as heart and lung failure.

An individual addicted to a drug of abuse will generally seek out that drug and take it because it provides a familiar and pleasurable rush. If a user were to take the drug and not experience the rush, it is believed that the addiction could be more easily broken. It is therefore believed that as part of a treatment regimen to end the addiction, it would be beneficial to remove, suppress, or otherwise deactivate the drug molecules shortly after they are introduced to the body. Additionally, a technique for specifically removing drug molecules from a patient's system could be useful in treating acute drug overdoses. Further, it would be beneficial to be able to limit the drug exposure of a fetus during pregnancy if the mother is a user of one or more drugs of abuse.

Owens, et al describe in published U.S. Patent Application 20010051158 one method of using involving monoclonal antibodies for treating medical problems associated with d-amphetamine-like drugs. However, the disclosed antibodies did not provide all of the desirable features for a treatment of drug addition, as described below. Accordingly, there is an unmet need in the art for a substance which is capable of selectively binding to a drug of abuse to deactivate it at a molecular level.

### SUMMARY OF THE INVENTION

One embodiment of the invention is a fully human monoclonal antibody that binds to drugs of abuse. In one embodiment, the monoclonal antibody has a heavy chain amino acid sequence as shown in Table 1. In another embodiment, the antibody further comprises a light chain amino acid sequence shown in Table 2. In preferred embodiments, the drugs of abuse are amphetamine, methamphetamine, or phencyclidine.

Another embodiment of the invention is a fully human antibody that binds to drugs of abuse that comprises a heavy chain amino acid sequence comprising the Complementarity Determining Regions (as defined by Kabat et al., Sequences of Proteins of Immunological Interest, Fifth Edition, NIH Publication 91-3242, Bethesda Md. [1991], vols. 1-3) shown in Table 1.

Yet another embodiment of the invention is a fully human antibody that binds to drugs of abuse and comprises a light chain amino acid sequence having CDRs shown in Table 2.

It should be realized that embodiments of the invention are not limited to any particular form of an antibody. For example, the anti-drug antibody may be a full length antibody (e.g having an intact human Fc region) or an antibody fragment (e.g a Fab, Fab′ or F(ab′)₂). In addition, the antibody may be manufactured from a hybridoma that secretes the antibody, or from a recombinantly produced cell that has been transformed or transfected with a gene or genes encoding the antibody.

Other embodiments of the invention include isolated nucleic acid molecules encoding any of the antibodies described herein, vectors having an isolated nucleic acid molecule encoding the anti-amphetamine antibody, a host cell transformed with such a nucleic acid molecule. In addition, one embodiment of the invention is a method of producing an anti-drug antibody by culturing host cells under conditions wherein a nucleic acid molecule is expressed to produce the antibody followed by recovering the antibody from the host cell.

Other embodiments of the invention include a pharmaceutical composition comprising an effective amount of the antibody of the invention in admixture with a pharmaceutically acceptable carrier.

In a different aspect, the invention includes a method for diagnosing a condition associated with the presence of a drug of abuse in a cell, comprising contacting the cell with an anti-drug antibody, and detecting the presence of the drug.

In yet another aspect, the invention includes a method for treatmenting addiction or other conditions associated with the use of drugs of abuse by a patient, comprising administering to the patient an effective amount of an anti-drug antibody.

In a further aspect, the invention includes a method of reducing the drug exposure to a fetus during pregnancy wherein the mother is a user of one or more drugs of abuse.

In another aspect, the invention includes a method of using anti-drug antibodies to counteract the effects of a drug of abuse on a patient or in treating a drug overdose.

Yet another embodiment of the invention is an article of manufacture, or a kit, that includes a container having a composition containing an antibody against a drug of abuse, and a package insert or label indicating that the composition can be used to treat addiction to a drug of abuse. The antibody may specifically bind to drugs of abuse such as amphetamines, methamphetamines, or phencyclidines.

### BRIEF DESCRIPTION OF THE DRAWINGS

FIG. 1 shows equilibrium dialysis data on Cell Culture Supernatants for anti-amphetamine antibodies.

FIG. 2 shows equilibrium dialysis data on Cell Culture Supernatants for anti-amphetamine antibodies.

FIG. 3 shows equilibrium dialysis data on Cell Culture Supernatants for anti-amphetamine antibodies and shows labeled amphetamine binding inhibition.

FIG. 4 shows equilibrium dialysis data on Cell Culture Supernatants for anti-amphetamine antibodies.

### DETAILED DESCRIPTION OF THE PREFERRED EMBODIMENT

Embodiments of the invention relate to fully human antibodies, or binding fragments thereof, that are useful as treatments for addiction to drugs of abuse. In one embodiment, the antibodies are useful to treat amphetamine or methamphetamine addiction. The antibodies are preferably given to a person suffering from addiction to a drug of abuse, wherein the antibody reduces the body's ability to metabolize the drug. This interference reduces the pleasurable sensation associated with the drug of abuse, thereby reducing an addicts craving for the drug. In addition, the antibody preferably helps clear the drug of abuse from the person's body before it can damage any internal organs. Thus, the antibodies are useful in acute situations, such as when a person is suffering from an overdose of such a drug.

Nucleotide and translated amino acid sequences of exemplary antibodies against amphetamines are set forth in Tables 1, 2, 3, and 4. Table 1 shows an analysis by class for the heavy chain sequence of an anti-amphetamine antibody. Table 2 shows an analysis by class for the light chain sequence of an anti-amphetamine antibody. Table 3 shows a project summary for the heavy chain of an anti-amphetamine antibody. Table 4 shows a project summary for the light chain of an anti-amphetamine antibody. The various chain names are identified in the left column of these figures. In these chain names, UA002 refers generally to an antibody against amphetamine. Of the other letters in the name of the chain, H refers to a heavy chain and K refers to a kappa chain. The numerical identifiers (as in 12_{—}5_{—}1) refer to the fusion number and the sample numbers (12.5.1, meaning fusion number 12). In particular, fusions 1, 2, and 3 are all G4 antibodies from Group 2. All of the fusion 12 antibodies are G2 antibodies from Group 1. The 36 fusion 13 antibodies (named 13.1 through 13.36) are from Group 3.

Additionally, nucleic acids encoding antibodies against drugs of abuse, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully in the following disclosure.

Furthermore, the proteins and polypeptides that make up antibodies against drugs of abuse, and fragments and variants thereof, may be used in ways that include (a) serving as an immunogen to stimulate the production of an anti-drug antibody, (b) a capture antigen in an immunogenic assay for such an antibody, (c) as a target for screening for substances that bind to drugs of abuse, and (d) a target for a drug-specific antibody such that the treatment induces a drug sink in which a drug of abuse can accumulate without exhibiting a physicokinetic effect on the patient. These utilities and other utilities for nucleic acids, polypeptides, antibodies, agonists, antagonists, and other related compounds are disclosed more fully below.

Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “drugs of abuse” or “drugs” refer to chemical agents which are either ingested or otherwise consumed by an individual and which may induce adverse health consequences. Drugs of abuse may or may not be regulated by government entities. The most common drugs of abuse include, for example, marijuana, cocaine, amphetamines, phencyclidine (PCP), heroin, hallucinogens, alcohol, nicotine, prescription medications, steroids, and inhalants.

An antibody that has “specifically binding” for an antigen or “specifically binds with” an antigen is an antibody with a strong preference for the specified antigen. An antibody that specifically binds with an antigen may also bind at a low level to other unrelated antigens.

The term “hapten” as used herein shall mean a small molecule that can react with a specific antibody but cannot generally induce the formation of antibodies unless bound to a carrier protein or other large antigenic molecule. The term “hapten” may also refer to a particular antigen, a modified antigen, or an analog of an antigen. “Hapten” can refer to a small molecule as described above whether or not it is or will ever be coupled to a carrier protein.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as -, -disubstituted amino acids, N-alkyl amino acids, lactic acid, and other unconventional amino acids may also be suitable components for polypeptides of the present invention. Examples of unconventional amino acids include: 4-hydroxyproline, -carboxyglutamate, —N,N,N-trimethyllysine, —N-acetyllysine, O-phosphoserine, N-acetylserine, N-formylmethionine, 3-methylhistidine, 5-hydroxylysine, —N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a drug of abuse, under suitable binding conditions, (2) ability to block appropriate drug binding, or (3) ability to inhibit a drug's chemical activity in vitro or in vivo. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of:—CH2NH—, —CH2S—, —CH2—CH2—, —CH═CH—(cis and trans), —COCH2—, —CH(OH)CH2—, and —CH2SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is 1 M, preferably 100 nM and most preferably 10 nM.

The term “agent” is used herein to denote a chemical compound, a mixture of chemical compounds, a biological macromolecule, or an extract made from biological materials.

“Mammal” when used herein refers to any animal that is considered a mammal. Preferably, the mammal is human.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites.

“Fab” when used herein refers to a fragment of an antibody which comprises the constant domain of the light chain and the CH1 domain of the heavy chain.

“Liposome” when used herein refers to a small vesicle that may be useful for delivery of drugs that may include the antibodies or immunoconjugates of the present invention.

“Label” or “labeled” as used herein refers to the addition of a detectable moiety to a polypeptide, for example, a radiolabel, flourescent label, enzymatic label chemiluminescent lable or a biotinyl group. Radioisotopes or radionuclides may include 3H, 14C, 15N, 35S, 90Y, 99Tc, 111In, 125I, 131I, fluorescent labels may include rhodamine, lanthamide phosphors or FITC and enzymatic labels may include horseradish peroxidase, -galactosidase, luciferase, alkaline phosphatase.

The term “pharmaceutical agent or drug” as used herein refers to a chemical compound or composition capable of inducing a desired therapeutic effect when properly administered to a patient. Although the term “drug” is also used in the present disclosure with respect to “drugs of abuse,” those of skill in the art will recognize that it is not necessary to distinguish between drugs which are administered to induce a medically desired therapeutic effect and those which are taken for another reason. Indeed, the same compound may be a drug of abuse or a therapeutic agent depending on its context.

Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g, Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Human Antibodies and Humanization of Antibodies

Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of human antibody function into a rodent so that the rodent produces fully human antibodies.

One method for generating fully human antibodies is through the use of XenoMouse™ strains of mice which have been engineered to contain 245 kb and 190 kb-sized germline configuration fragments of the human heavy chain locus and kappa light chain locus. See Green et al. Nature Genetics 7:13-21 (1994). The XenoMouse strains are available from Abgenix, Inc. (Fremont, Calif.).

The production of the XenoMouse is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430, 938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B 1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against drugs of abuse in order to vitiate concerns and/or effects of HAMA or HACA response.

Additional Criteria for Antibody Therapeutics

As discussed herein, the function of an anti-drug antibody appears important to at least a portion of its mode of operation. By function, we mean, by way of example, the activity of the anti-drug antibody in binding to a drug molecule. Accordingly, in certain respects, it may be desirable in connection with the generation of antibodies as therapeutic candidates against the drug that the antibodies be capable of fixing complement and participating in CDC. There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, one of the anti-drug antibodies discussed herein is a human anti-amphetamine IgG2 antibody. Another anti-drug antibody discussed herein is a human anti-amphetamine IgG4 antibody. If such antibody possessed desired binding to the amphetamine molecule, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC.

Accordingly, as antibody candidates are generated that meet desired “structural” attributes as discussed above, they can generally be provided with at least certain of the desired “functional” attributes through isotype switching.

Design and Generation of Other Therapeutics

In accordance with the present invention and based on the activity of the antibodies that are produced and characterized herein with respect to drugs of abuse, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation is a desirable attribute, it may be possible to sidestep the dependence on complement for cell killing through the use of bispecifics, immunotoxins, or radiolabels, for example.

For example, in connection with bispecific antibodies, bispecific antibodies can be generated that comprise (i) two antibodies one with a specificity to a drug of abuse and another to a second molecule that are conjugated together, (ii) a single antibody that has one chain specific to a drug of abuse and a second chain specific to a second molecule, or (iii) a single chain antibody that has specificity to a drug of abuse and the other molecule. Such bispecific antibodies can be generated using techniques that are well known for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)).

In connection with immunotoxins, antibodies can be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902.

Therapeutic Administration and Formulations

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (18th ed, Mack Publishing Company, Easton, Pa. (1990)), particularly Chapter 87 by Block, Lawrence, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Preparation of Antibodies

Antibodies in accordance with the invention were prepared through the utilization of the XenoMouse technology, as described below. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the Background, herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through use of such technology, we have produced fully human monoclonal antibodies to a variety of antigens. Essentially, we immunize XenoMouse™ lines of mice with an antigen of interest, recover lymphatic cells (such as B-cells) from the mice that expressed antibodies, and fuse such recovered cell lines with a myeloid-type cell line to prepare immortal hybridoma cell lines, and such hybridoma cell lines are screened and selected to identify hybridoma cell lines that produced antibodies specific to the antigen of interest. Herein, we describe the production of multiple hybridoma cell lines that produce antibodies specific to drugs of abuse. Specific examples disclosed herein include antibodies specific to amphetamine, methamphetamine, and phencyclidine. Further, we provide a characterization of the antibodies produced by such cell lines, including nucleotide and amino acid sequence analyses of the heavy and light chains of such antibodies.

Alternatively, instead of being fused to myeloma cells to generate hybridomas, the recovered cells, isolated from immunized XenoMouse™ lines of mice, are screened further for reactivity against the initial antigen, preferably amphetamine, methamphetamine, or phencyclidine.

Such screening includes an ELISA, a competition assay with known antibodies that bind the antigen of interest. Using reverse-transcriptase PCR, the DNA encoding the variable region of the antibody secreted can be cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, preferably CHO cells, and cultured in conventional nutrient media modified as appropriate for inducing promoters, selecting transformants, or amplifying the genes encoding the desired sequences. Herein, we describe the isolation of multiple single plasma cells that produce antibodies specific to a drug of abuse. Further, the genetic material that encodes the specificity of the anti-drug antibody is isolated, introduced into a suitable expression vector which is then transfected into host cells.

As will be appreciated, antibodies in accordance with the present invention can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive drug binding properties.

Antibodies in accordance with the present invention are capable of binding to a drug of abuse. Further, antibodies of the invention are useful in the detection of a drug of abuse in patient samples and accordingly are useful as diagnostics as described hereinbelow. In addition, based on the potent inhibition of growth of fibroblast cells observed through use of antibodies of the invention, it is expected that such antibodies will have therapeutic effect in the treatment of drug addiction, drug abuse, and drug overdose as discussed hereinbelow.

### EXAMPLES

The following examples, including the experiments conducted and results achieved are provided for illustrative purposes only and are not to be construed as limiting upon the present invention.

### EXAMPLE 1

### Haptens

One hapten that is useful in raising antibodies against amphetamine is (S)-(+)-4-(5-Carboxypentyloxy) amphetamine HCl. This hapten has a molecular weight of 310.82 and is hereinafter referred to as Amp(+)MO6. In preferred embodiments, this hapten is attached to a carrier molecule such as Bovine Serum Albumin, Keyhole Limpet Hemocyanin (KLH), or another large molecule which is capable of inducing an antibody response.

### EXAMPLE 2

### Antibody Structures

Examples of amino acid sequences of anti-amphetamine antibodies are disclosed in Tables 1 and 2. Nucleotide sequences of anti-amphetamine antibodies are shown in Tables 3 and 4. Table 1 shows the amino acid sequence for a series of heavy chain variable regions of anti-amphetamine antibodies. Table 2 shows the amino acid sequences of for a series of light chain variable domains of anti-amphetamine antibodies. Table 3 shows the nucleotide sequence for the heavy chain variable regions of a series of anti-amphetamine antibodies. Table 4 shows the nucleotide sequences for the light chain variable regions of a series of anti-amphetamine antibodies.

### EXAMPLE 3

### Synthesis of Hapten-Protein Conjugate

Each drug-like hapten was covelantly bound to a protein by a direct reaction using carbodiimide as the coupling agent (1-step procedure). For this synthesis, a six-fold molar excess of hapten was added and 10×EDCI as compared to the protein. See “A Simple Modified Carbodiimide Method for Conjugation of Small Molecular-Weight Compounds to Immunoglobulin G with Minimal Protein Crosslinking.”, Davis and Preston, Anal Biochem, 116, 402-407, 1981; Owens et al., JPET, Vol. 246, No. 2, p. 472-478, 1988. In this experiment, a KLH conjugate was used to conjugate to the amphetamine hapten.

The following abbreviations apply to this experiment:
Amp(+)MO6 refers to (S)-(+)-4-(5-Carboxypentyloxy) amphetamine HCl, m.w. =310.82 mg/mM.
KLH refers to Inject Keyhole Limpet Hemocyanin, m.w. =6,700,000 g/M or mg/mM (Inject Keyhole Limpet Hemocyanin, Product #77100, Pierce, Rockford, Ill.).
DMF refers to N,N-Dimethyl Formamide, few. 73.09 g/M (ACS Reagent, Product #D-8654, Sigma Chemical Company, St. Louis, Mo.).
EDCI refers to 1-Ethyl-3-(3-Dimethylamino propyl carbodiimide), m.w.=191 g/M or mg/mM (Product # E-7750, Sigma Chemical Company, St. Louis, Mo.).

The amount of KLH needed was calculated as follows: It was assumed that there were 600 free lysine molecules on the protein (KLH) which are available for hapten/protein covalent binding formation. Since we wanted to use 6 fold excess of moles of hapten per mole protein, we chose 3600 moles of hapten per mole protein: Starting with 20 mg of KLH: (20 mg KLH)/(6,700,000 mg/mM)=0.00000299 mM or 2.99 nM. Therefore, 3600 moles hapten/l mole KLH×2.99 nM KLH=10,764 nM or 10.76 μM hapten needed for a 3600:1 ratio of hapten to KLH protein.

The amount of hapten needed for KLH coupling was calculated as follows: (10.76 μM hapten)×(310.82 μg/μM)=3344 μg or 3.34 mg of AMP(+)M06 hapten for 3600:1 ratio.

The amount of EDCI needed was calculated as follows: We needed 10×excess of EDCI as the amount of hapten present in the reaction to block excess sites. Accordingly: (10)×(10.76 μM)=107.6 μM; (107.6 μM)×(191 μg/μM)=20,551 μg or 20.6 mg EDCI.

The following method was carried out to synthesize the amphetamine/KLH conjugate. All buffers and reagents were kept at room temperature. The KLH protein was dissolved in 1 ml of water and then brought to a final concentration of 0.166 M Sodium Phosphate, 1.8 M Sodium Chloride, pH 7.2. This solution was dialysed 0.1 M Mes, pH 4.5, overnight. (against 2L Mes in 10K Slide-A-Lyzer).

The hapten was dissolved in 500 μl DMF, followed by addition of 1.0 mL deionized H2O. The pH was then adjusted to 4.5 with the addition of dilute HCl (10 μl of 1:10 dilution of concentrated HCl). The hapten and the protein solutions were combined slowly with stirring by adding the protein to the hapten solution. They were then activated for 5 minutes at room temperature. The EDCI was dissolved in 100 μl of 0.1 M Mes, pH 4.5, while keeping the EDCI protected from light. The EDCI solution was slowly added to the hapten/cBSA solution while mixing. The mixture was left to react overnight at RT with continued mixing and protection from light.

The bound hapten was separated from the free hapten using a Sephadex 25 (P10) column from Pierce. The column was then equilibrated with sterile PBS, and the sample applied while 1 mL fractions were collected. Each sample was run through a spectrometer to determine which fraction contained the protein bound to hapten. Each fraction with protein bound to hapten was combined and concentrated to no less than 1 mg/mL.

A similar protocol was carried out to generate the BSA/hapten conjugations.

### EXAMPLE 4

### Immunization of Mice

Drug haptens were employed as an immunogen to stimulate an immune response in XenoMouse® animals (Abgenix Inc, Fremont, Calif.). Specifically, the drugs against which the antibodies were raised were amphetamine, methamphetamine, and phencyclidine. Monoclonal antibodies directed against the drugs of abuse were prepared by hybridoma technology from immunized XenoMouse animals in standard fashion.

Table 5 shows an immunization schedule in which mice were immunized with the various immunoconjugates. UA001 refers to an immunoconjugate of a methamphetamine hapten bound to BSA, UA002 refers to an immunoconjugate of an amphetamine hapten bound to BSA, UA003 refers to an immunoconjugate of a phencyclidine (PCP) hapten bound to BSA. For each immunoconjugate, two groups of mice were used; group 1 contained Xenomouse xmg2 strain mice (Abgenix, Inc., Fremont Calif.) while group 2 contained Xenomouse 3C-1 strain mice (Abgenix, Inc., Fremont Calif.).

Table 6 shows a similar immunization schedule in which mice were immunized with various immunoconjugates. Here, UA002 refers to an immunoconjugate of an AMP(+)MO6-KLH, UA003 refers to an immunoconjugate of PCHAP/KLH. For the study of these immunoconjugates, a third group of mice (group 3) was used; group 3 mice were xmg2 strain mice.

Table 7 shows titer data illustrating the Xenomouse response in group 1 to the innoculation with the amphetamine-BSA immunoconjugate. The mouse ID numbers are shown in the left colmun; NC(h) refers to a negative control (human), NC(m) refers to negative control (mouse), and PC(m) refers to positive control (mouse).

Table 8 shows titer data illustrating the Xenomouse response in group 2 to the innoculation with the amphetamine-BSA immunoconjugate. The mouse ID numbers are shown in the left colmun; NC(h) refers to a negative control (human), NC(m) refers to negative control (mouse), and PC(m) refers to positive control (mouse).

Table 9 shows titer data illustrating the Xenomouse response in group 3 to the innoculation with the AMP(+)MO6-KLH immunoconjugate. The mouse ID numbers are shown in the left colmun; NC refers to a negative control and PC refers to a positive control.

Table 10 shows sequence information corresponding to various clones used in the UA002 project.

### EXAMPLE 5

### ELISA Protocol

The following example is an enzyme-linked immunosorbent assay (ELISA) protocol used to detect the presence of antibody in cell culture supernatant or serum.

The following reagents were used: Alkaline Phosphatase labeled secondary antibody (Sigma, Cat. # A-1418); Coating Buffer (See REAGENT PREPARATION); Diethanolamine, C4H11NO₂ (Sigma, Cat. # D-8885); ELISA Wash Buffer; Hydrochloric Acid, HCl (Sigma, Cat. # H7020); Magnesium Chloride, MgCl2.H₂O (Sigma, Cat. # M-2393); Potassium Chloride, KCl (Fisher, Cat. # P330-500); Potassium Phosphate Monobasic, KH2PO₄ (Sigma, P-5379); p-Nitrophenyl Phosphate Tablets (Sigma, Cat. # N-2640, Lot # 49H8252); 1×PBS; 10×PBS; Sodium Bicarbonate, NaHCO3 (Fisher, Cat. # S-233); Sodium Carbonate, Na2CO3 (Fisher, Cat. # S-263); Sodium Chloride, NaCl (Fisher, Cat. # BP358-212); Sodium Phosphate Dibasic, Na2HPO4 (J T Baker, Cat. # 4062-01); Substrate; Substrate Buffer; Superblock Blocking Buffer (Pierce, Cat. # 37517); Tween 20, C58H114O26 (Fisher, Cat. # BP337-500).

Reagent Preparation

ELISA Wash Buffer: Transfer 400 ml 10×PBS solution, to a 4L plastic beaker. Add 4 ml Tween 20 detergent, and bring up to 4L with MilliQH2O. Add stir bar, place beaker on stir plate, and mix well by allowing it to stand under constant stirring for at least 2 minutes. Transfer to clean, labeled 4L bottle with cap, and store in 4° C. refrigerator.

Substrate Buffer: Transfer 48 ml diethanolamine to a 500 ml beaker. Add 0.05 g MgCl2.6H2O. Add approximately 400 ml MilliQH₂O, and adjust pH to 9.0 with 1 N HCl. Bring volume up to 500 ml with MilliQ H₂O. Transfer to clean, labeled bottle and store in 4° C.

Substrate: Under constant stirring, add 30 mg (or 2 tablets) p-Nitrophenyl Phosphate/50 ml Substrate Buffer. For 500 ml Substrate Buffer, add 20 tablets of p-Nitrophenyl Phosphate. Allow the tablets to dissolve and then aliquot 12 ml/tube into sterile, labeled 15 ml conical centrifuge tubes. Store tubes at −20° C.

Procedure

Dilute antigen in coating buffer, generally to 100 ng/well. See sample calculation below. Add 100 μl/well of antigen in coating buffer to 96-well ELISA plate. Place plates in a plastic Tupperware container with lid. Place a moistened paper towel in the bottom to ensure a humid environment for the plates, and place container in 37° C. incubator for 3 hours.

Remove coating buffer and wash plate with ELISA wash buffer on ELISA plate washer. (200 μl/well, 5 times). Remove excess wash buffer, and strike the plate several times on paper towels to remove any excess wash buffer. Add 180 μl/well of Superblock Blocking Buffer. Incubate plates in humid container at 37° C. for maximum of 1 hour. Remove excess buffer, and store the plates in a sealed moist chamber at 4° C. until ready to use.

Prepare desired dilutions of 1° antibody in ELISA Wash Buffer containing 10% Superblock Blocking Buffer. Generally, undiluted cell culture supernatant is used. Add 100 μl per well. Incubate in moist container at room temperature for 3-4 hours or overnight at 4° C.

Wash plate with ELISA Wash Buffer, using the ELISA plate washer (200 μl/well, 5 times). Remove excess wash buffer, and strike the plate several times on paper towels to remove any excess wash buffer. Prepare a 1:6000 dilution of alkaline phosphatase labeled secondary antibody in ELISA Wash Buffer containing 10% Superblock Blocking Buffer, by mixing the following, for each plate: 1.6 μl alkaline phosphatase labeled secondary antibody; 1 ml Superblock Blocking Buffer; and 9 ml ELISA wash buffer. Add 100 μl/well. Incubate at room temperature for 1 hour in a moist chamber.

While the plate is in incubation, allow the substrate to thaw, approximately 11 ml per plate. Using the ELISA plate washer, wash the plate with ELISA wash buffer. (200 μl/well, 5 times). Remove excess wash buffer, and strike the plate several times on paper towels to remove excess wash buffer. Add 100 μl Substrate to each well. Incubate at 37° C. for approximately 1 hour. The amount of time needed for development may vary. An optimal optical density occurs when the most developed well reaches at least 1.5 or higher. Check the plate at 15 minute intervals so that the development may be tracked.

Read the plate with an ELISA plate reader at 405-410 nm filter.

Sample Calculation

The following calculations are used to determine the amount of reagents that should be used: Diluting antigen for coating ELISA plates: Stock Antigen: PCHAP/oval 1.6 mg/ml=1.6 μg/μl. Each plate should be coated with 100 ng Antigen/well. Each plate has 96 (or approximately 100) wells/plate: (100 ng Antigen/well)×(100 wells/plate)=10 μg Antigen/plate. Each plate requires 10 ml of coating buffer: (10 μg Antigen/plate)/(1.6 μg/μl)=6.25 μl/plate (in 10 ml coating buffer).

### EXAMPLE 6

### Equilibrium Dialysis Experiment

The following protocol provides a method to determine whether anti-amphetamine antibodies prepared as described above are capable of binding to amphetamine target molecules.

Drug or hapten protein binding in tissue culture media was determined by equilibrium dialysis as previously described (Valentine and Owens, 1996). Dialysis discs with a molecular weight cutoff of 3,500 (Spectrum Medical Industries Inc., Los Angeles, Calif.) were placed in Teflon dialysis cells (Spectrum Medical Industries Inc., Los Angeles, Calif.). Tissue culture media from potentially positive antibody cell lines were spiked with a tracer amount of radiolabeled drug (e.g., [3H]AMP at about 100,000 dpm per sample) and placed in one side of the equilibrium dialysis chamber. Phosphate buffer (120 1, 0.13 M, pH 7.4) was added to the opposite side of the dialysis chamber. The dialysis cells were incubated overnight using constant rotation at room temperature. The protein (tissue culture media) and buffer samples were removed from the dialysis chamber, and the radioactive drug/hapten concentrations were determined in each side by liquid scintillation spectrometry. The fraction of unbound radiolabeled drug/hapten was calculated by dividing the unbound dpm in the buffer side by the total dpm in the antibody supernatant side.

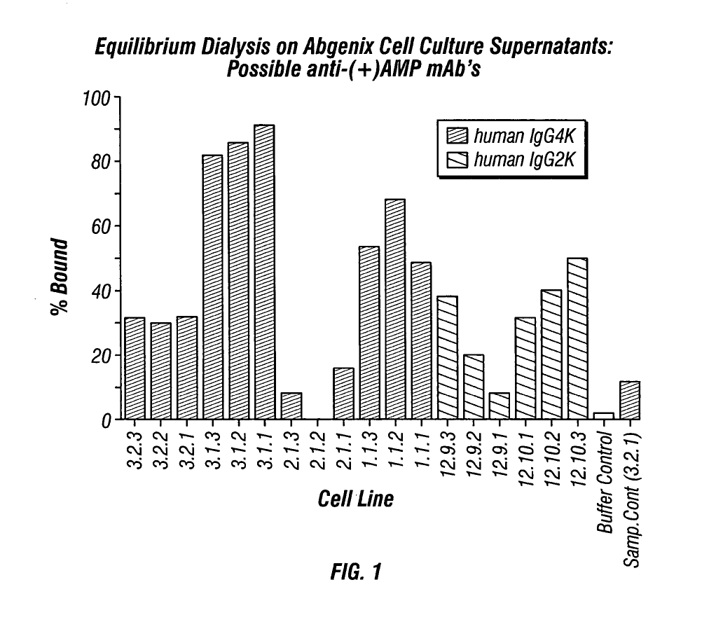

The results are shown in FIGS. 1, 2, 3, and 4.

FIG. 1 shows equilibrium dialysis data on Cell Culture Supernatants for a first set of anti-amphetamine antibodies.

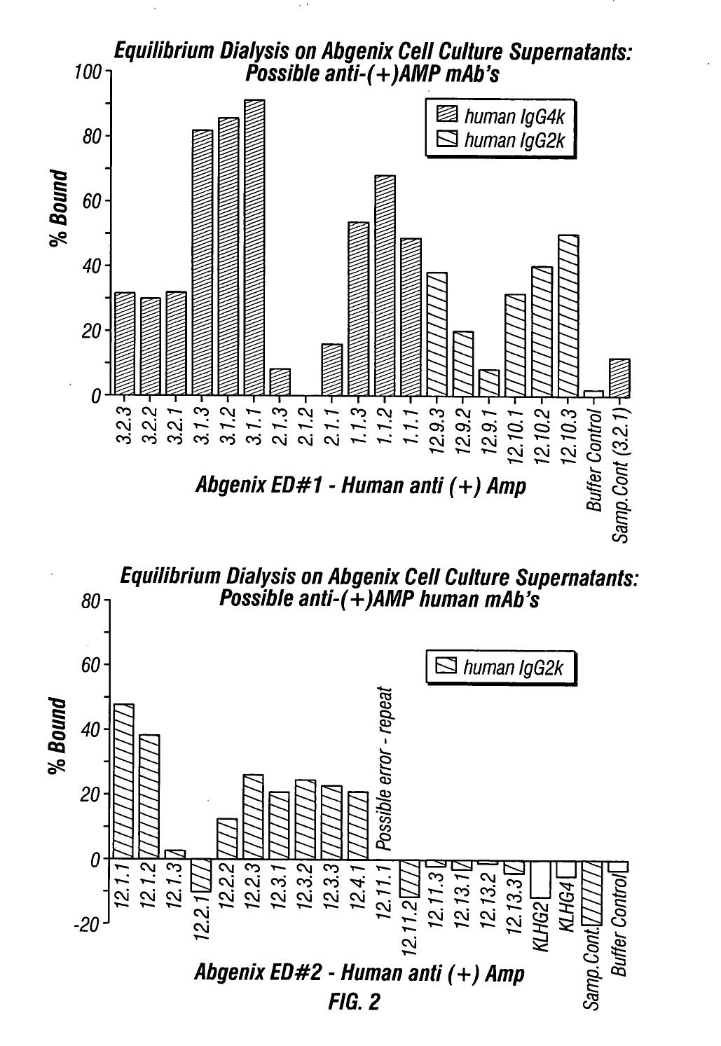

FIG. 2 shows equilibrium dialysis data on Cell Culture Supernatants for a second set of anti-amphetamine antibodies.

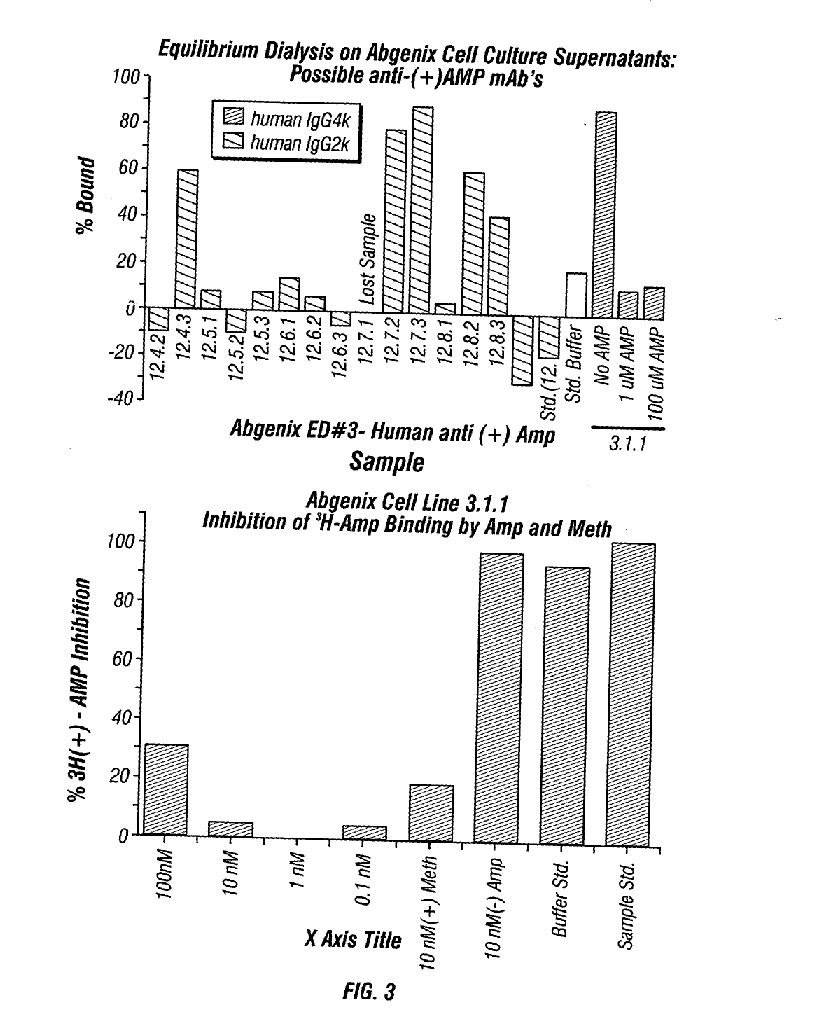

The first graph of FIG. 3 shows equilibrium dialysis data on Cell Culture Supernatants for anti-amphetamine antibodies. The second graph of FIG. 3 shows binding inhibition of amphetamine radiolabeled with 3H by cold amphetamine and cold methamphetamine.

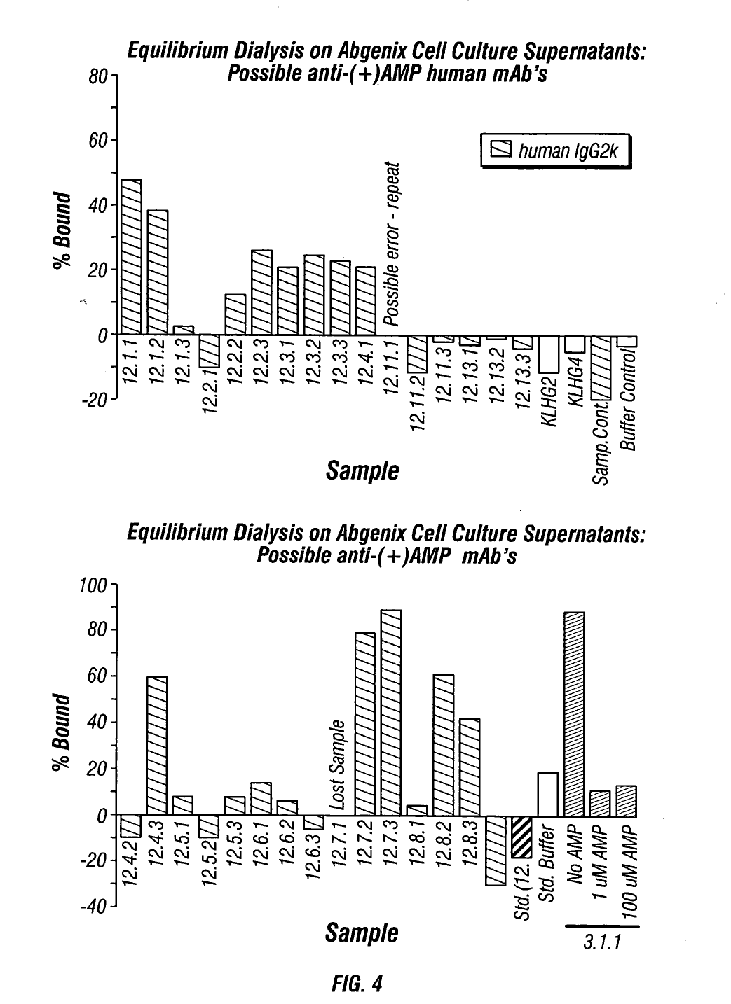

FIG. 4 shows equilibrium dialysis data on Cell Culture Supernatants for a third set of anti-amphetamine antibodies.

### EXAMPLE 7

### Radioimmunoassay

The following Radioimmunoassay (RIA) was used to determine the Kd value for AMP(+)MO6 human clones (exhaustion supernatant from Abgenix) using cold (+)AMP at various concentrations. These clones are from UA002 Fusions 3, 12, & 13.

The following reagents were added in order to each sample tube by pipettor diluter: 10 μl of cold drug or RIA buffer for NSB and Bo controls; 100 μl of [±3H]AMP in 2% BSA-RIA buffer at 80,000 dpm/tube; 100 μl of anti-AMP mAb or negative control sample for the NSB controls.

The samples were incubated overnight at 4° C. Then, the samples were harvested with 500 μl per tube of 1% Goat anti-Human, kappa specific 2nd Antibody in 6% PEG-RIA buffer. The harvested material was then vortexed.

The harvested material was then incubated for 20 min at 4° C., centrifuged for 20 min at. 4° C. at 3800 rpm. The supernatant was aspirated. 2 ml of EcoScint, a scintillation fluid, was added, and the samples were vortexed. The samples were then allowed to sit for 1 hour at room temperature, vortexed, and placed in sample tubes in 20 ml scintillation vials, capped, and counted for 5 min each at 2% efficiency.

### EXAMPLE 8

### Treating Patients with Anti-Amphetamine Antibodies

As follows, anti-amphetamine antibodies can be administered to patients for the purpose of binding and/or deactivating drug molecules. Preferably, such treatment is part of a rehabilitation program; the delivery of antibodies can create a “drug sink” in the body of the patient such that when new drug molecules enter the body, antibodies already in the system can bind and/or deactivate them. Alternatively, antibodies can be given to counteract the effect of drugs already in the system, as in a treatment for a drug overdose. In such cases, it is preferable that the antibodies be administered promptly after the drug of abuse has been introduced to the system.

To determine the in vivo effects of anti-amphetamine antibody treatment in human patients addicted to amphetamine, such human patients are injected twice per day with approximately 5 mg/kg of body weight with an antibody containing a sequence as described in Table 1 and/or Table 2. The patient is monitored daily to determine the amount of amphetamine in the blood stream or other tissue and whether the patient exhibits typical signs and symptoms associated with amphetamine addiction. The patient treated with anti-amphetamine antibodies is found to have a lower level of free amphetamine in the body compared to that of similar amphetamine-addicted patients treated with control antibodies.

Control antibodies that may be used include antibodies of the same isotype as the anti-amphetamine antibodies tested but do not have the ability to bind to amphetamine. Further, in the patient treated with anti-amphetamine antibodies, the symptoms of amphetamine addiction diminish as treatment proceeds. Upon the conclusion of the treatment regimen, the patient treated with anti-amphetamine antibodies is no longer addicted to amphetamine.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents.

### Incorporation by Reference

All references cited herein, including patents, patent applications, papers, text books, and the like, and the references cited therein, to the extent that they are not already, are hereby incorporated herein by reference in their entirety. In addition, the following references are also incorporated by reference herein in their entirety, including the references cited in such references:

### Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

TABLE 1
Project: UA002

| Seq. | | | | | | | |
|---|---|---|---|---|---|---|---|
| ID NO. | Chain Name | V | D | J | FR1 | CDR1 | FR2 |
| 1 | | | Germline | | EVQLVESGGGLVKPGGSLRLSCAAS | GFTFSNAWMS | WVRQAPGKGLEWVG |
| 2 | UA002H12_5_1N1G2 | VH3-15 | D2-15 | JH6b | EVQLVESGGGLVKPGGSLRLSCAAS | GFTFNNAWMS | WVRQAPGKGLEWVG |
| 3 | | | Germline | | QVQLVESGGGVVQPGRSLRLSCAAS | GFTFSSYGMH | WVRQAPGKGLEWVA |
| 4 | UA002H3_1_1NIG4 | VH3-33 | D3-16 | JH3b | QVQLVESGGGVVQPGRSLRLSCAAS | GFSFINYGMH | WVRQAPGKGLEWVA |
| 5 | UA002H1_1_1N1G4 | ′ | ′ | ′ | QVQLVESGGGVVQPGRSLRLSCAAS | GFTFSSYGMH | WVRQAPGKGLEWVA |
| 6 | | | Germline | | EVQLLESGGGLVQPGGSLRLSGAAS | GFTFSSYAMS | WVRQAPGKGLEWVS |
| 7 | UA002H12_7_1N1G2 | VH3-23 | D5-12 | JH6b | EVQLLESGGGLVQPGGSLRLSCAAS | GFTFSNYAMS | WVRQAPGKGLEWVS |
| 8 | | | Germline | | EVQLVESGGGLVKPGGSLRLSCAAS | GFTFSNAWMS | WVRQAPGKGLEWVG |
| 9 | UA002H12_9_1N1G2 | VH3-15 | D3-16 | JH4b | EVQLVESGGGLVKPGGSLRLSCAAS | GFTFSNAWMS | WVRQAPGKGLEWVG |
| 10 | UA002H12_3_1N1G2 | ′ | ′ | ′ | EVQLVESGGGLVKPGGSLRLSCAAS | GFTFSNALMS | WVRQAPGKGLEWVG |
| 11 | UA002H12_1_IN1G2 | ′ | ′ | ′ | EVQLVESGGGLVKPGGSLRLSCAAS | GFTFSNAWMS | WVRQAPGKGLEWVG |
| 12 | | | Germline | | QVQLVESGGGVVQPGRSLRLSCAAS | GFTFSSYGMH | WVRQAPGKGLEWVA |
| 13 | UA002H12_4_1N1G2 | VH3-33 | D6-19 | JH4b | QVQLVESGGGVVQPGRSLRLSCAAS | GFTFNTYVMH | WVRQAPGKGLEWVA |
| 14 | UA002H12_6_1N1G2 | ′ | ′ | ′ | QVQLVESGGGVVQPGRSLRLSCAAS | GFTFSSYGMH | WVRQAPGKGLEWVA |
| 15 | UA002H12_8_1N1G2 | ′ | ′ | ′ | QVQLVESGGGVVQPGRSLRLSCAAS | GFTFNTYVMH | WVRQAPGKGLEWVA |
| 16 | UA002H12_13_1N1G2 | ′ | ′ | ′ | QVQLVESGGGVVQPGRSLRLSCAAS | GFTFNTYVMH | WVRQAPGKGLEWVA |
| 17 | | | Germline | | EVQLVESGGGLVKPGGSLRLSCAAS | GFTFSSYSMN | WVRQAPGKGLEWVS |
| 18 | UA002H2_1_1N1G4 | VH3-21 | D6-19 | JH6b | EVQLVESGGGLVKPGGSLRLSCAAS | GFTFSSYTMN | WVRQAPGKGLEWVS |
| 19 | UA002H12_11_1N1G2 | ′ | ′ | ′ | EVQLVESGGGLVKPGGSLRLSCAAS | GFTFSSYCMN | WVRQTPGKGLEWVS |
| 20 | | | Germline | | EVQLLESGGGLVQPGGSLRLSCAAS | GFTFSSYAMS | WVRQAPGKGLEWVS |
| 21 | UA002H12_2_1N1G2 | VH3-23 | | JH6b | EVQLLESGGGLVQPGGSLRLSCAAS | GFTFSSYAMS | WVRQAPGKGLEWVS |

| Seq. | | | | | |
|---|---|---|---|---|---|
| ID No. | Chain Name | CDR2 | FR3 | CDR3 | J |
| 1 | | RIKSKTDGGTTDYAAPVKG | RFTISRDDSKNTLYLQMNSLKTE | ###CSGGS###YYYYGMDV | WGQGTTVTVSSA |
| | | | DTAVYYCTT | | |
| 2 | UA002H12_5_1N1G2 | RIKSKIDGGTTDYAAPVKG | RFTMSRDDSKNTLYLQMNSLKTE | DEDCSGGSCFFYHYYLMDV | WGQGTTVTVSSA |
| | | | DTAVYYCTT | | |
| 3 | | VIWYDGSNKYYADSVKG | RFTISRDNSKNTLYLQMNSLRAE | ##YDYVWGSYRYT#AFDI | WGQGTMVTVSSA |
| | | | DTAVYYCAR | | |
| 4 | UA002H3_1_1N1G4 | VIWNDGRSKYYADSVKG | RFTISRDNSKNTLGLQMNSLRAE | EDYDYIWESFPYTGAFDI | WGQGTMVTVSSA |
| | | | DTAVYYCAR | | |
| 5 | UA002H1_1_1N1G4 | VIWNDGSYKYYADSVKG | RFTISRDNSKNTLYLQMNSLRAE | EDYDYDWMIDRYTGAFDL | WGQGTMVTVSSA |
| | | | DTAVYYCVR | | |
| 6 | | AISGSGGSTYYADSVKG | RFTISRDNSKNTLYLQMNSLRAE | #VDIVAT##YYYYGMDV | WGQGTTVTVSSA |
| | | | DTAVYYC## | | |
| 7 | UA002H12_7_1N102 | VISGSGGNTYYADSVRG | RFTISRDNSKNTLYLQMNSLRAE | HVDIVATMAYFDYVMDV | WGQGTTVTVSSA |
| | | | DTAVYYC## | | |
| 8 | | RIKSKTDGGTTDYAAPVKG | RFTISRDDSKNTLYLQMNSLKTE | #MITFGGVIVI#DY | WGQGTLVTVSSA |
| | | | DTAVYYCTT | | |
| 9 | UA002H12_9_1N1G2 | RIKSKTDGGTTDYAAPVKG | RFTISRDDSKNTLYLQMNSLKTE | EMITFGGVIVIPDY | WGQGTLVTVSSA |
| | | | DTAVYYCTT | | |
| 10 | UA002H12_3_1N1G2 | RIKSKTDGGTIDYAAPVKG | RFTISRDDSKNTLYLQMNSLKTE | #MITFGGVIVIPDY | WGQGTLVTVSSA |
| | | | DTAVYYCTT | | |
| 11 | UA002H12_1_1N1G2 | RIKSKTDGGTIDYVAPVKG | RFTISRDDSKNTLYLQMNSLKTE | EVITFGGIIVD#DY | WGQGTLVTVSSA |
| | | | DTAVYYCTT | | |
| 12 | | VIWYDGSNKYYADSVKG | RFTISRDNSKNTLYLQMNSLRAE | ##GIAVA###YFDY | WGQGTLVTVSSA |
| | | | DTAVYYCAR | | |
| 13 | UA002H12_4_1N1G2 | VIWYDGSNKKYADSVKG | RFTISRANSKNTLYLQMNSLRAE | DGGRTVADPYYFDY | WGQGTLVTVSSA |
| | | | DTAVYYCAR | | |
| 14 | UA002H12_6_1N1G2 | VIWYDGSNKNYADSVKG | RFTISRGNSKNTLYLQMNSLRAE | DGGIAVADPYYFDY | WGQGTLVTVSSA |
| | | | DTAVYYCAR | | |
| 15 | UA002H12_8_1N1G2 | VIWYDGSNKKYADSVKG | RFTISRANSKNTLYLQMNSLRAE | DGGRTVADPYYFDY | WGQGTLVTVSSA |
| | | | DTAVYYCAR | | |
| 16 | UA002H12_13_1N1G2 | VIWYDGSNKKYADSVKG | RFTISRANSKNTLYLQMNSLRAE | DGGRTVADPYYFDY | WGQGTLVTVSSA |
| | | | DTAVYYCAR | | |
| 17 | | SISSSSSYIYYADSVKG | RFTISRDNAKNSLYLQMNSLRAE | ####G##YYYYGMDV | WGQGTTVTVSSA |
| | | | DTAVYYCA# | | |
| 18 | UA002H2_1_1N1G4 | SISSSSSYIYYADSVKG | RFTISRDNAKNSLYLQMNSLRAE | DGGIGFYYYYYGMDV | WGQGTTVTVSSA |
| | | | DTAVYYCA# | | |
| 19 | UA002H12_11_1N1G2 | SISSSSYYIYYADSVKG | RFTISRDNAKNSLYLQMNSLRAE | DI##G#GGDYYGMDV | WGQGTTVTVSSA |
| | | | DTAVYYCA# | | |
| 20 | | AISGSGGSTYYADSVKG | RFTISRDNSKNTLYLQMNSLRAE | #######YYYYYGMDV | WGQGTTVTVSSA |
| | | | DTAVYYCAK | | |
| 21 | UA002H12_2_1N1G2 | SISGSGAYTYYADSVKG | RFTISRDNSKNTLYLQMNSLRAE | DLGEDEDYYDYYGVDV | WGQGTTVTVSSA |
| | | | DTAVYYCAK | | |

1. An isolated antibody, or binding fragment thereof, that specifically binds to a drug of abuse, and wherein said antibody comprises a heavy chain amino acid having a sequence selected from the group consisting of: SEQ. ID NO. 2, 4, 5, 7, 9, 10, 11, 13, 14, 15, 16, 18, 19, and 21.

2. The isolated antibody of claim 1, wherein said monoclonal antibody comprises a light chain amino acid having a sequence selected from the group consisting of: SEQ. ID NO. 23, 25, 26, 28, 30, 32, 33, 34, 35, 37, 38, 39, 41, and 43.

3. The antibody of claim 1 wherein the drug of abuse comprises amphetamine.

4. The antibody of claim 1 wherein the drug of abuse comprises methamphetamine.

5. The antibody of claim 1 wherein the drug of abuse comprises phencyclidine.

6. The antibody of claim 1, wherein the antibody is a monoclonal antibody.

7. The antibody of claim 1 wherein the antibody is a chimeric antibody.

8. The antibody of claim 1, wherein the antibody is a human antibody.

9. The antibody or fragment of claim 1, wherein the antibody or fragment is associated with a pharmaceutically acceptable carrier or diluent.

10. The antibody or fragment of claim 1, wherein the antibody or fragment is conjugated to a therapeutic agent.

11. The antibody or fragment of claim 10, wherein the therapeutic agent is a toxin.

12. The antibody or fragment of claim 10, wherein the therapeutic agent is a radioisotope.

13. A hybridoma cell line producing the antibody of claim 1.

14. A transformed cell comprising a gene encoding the antibody of claim 1.

15. The cell of claim 14, wherein the cell is a Chinese hamster ovary cell.

16. A method of treating a patient suffering from addiction to a drug of abuse comprising providing a therapeutically effective amount of an antibody of claim 1 to said patient.

17. The method of claim 16 wherein the drug of abuse comprises amphetamine.

18. The method of claim 16 wherein the drug of abuse comprises methamphetamine.

19. The method of claim 16 wherein the drug of abuse comprises phencyclidine.

20. An article of manufacture comprising a container, a composition contained therein, and a package insert or label indicating that the composition can be used to treat addiction to a drug of abuse, wherein the composition comprises the antibody of claim 1.

21. The article of manufacture of claim 20, wherein the drug of abuse is amphetamine or methamphetamine.
